(19) United States
(12) Patent Application Publication
(10) Pub. No.: US 2002/0188355 A1
(43) Pub. Date: Dec. 12, 2002

(54) Artificial knee joint with a joint friction member

(76) Inventor: Sen-Jung Chen, Taipei City (TW)

Correspondence Address: Ladas & Parry, Suite 1200, 224 South Michigan Avenue, Chicago, IL, 60604, US

(21) Appl. No.: 09/904,218

(22) Filed: Jul. 12, 2001

(30) Foreign Application Priority Data
Jun. 6, 2001 (TW) 090209399

(51) Int. Cl. A61F 2/64; A61F 2/68

(52) U.S. Cl. 623/45; 623/46

(57) ABSTRACT

An artificial knee joint includes an upper joint member, a lower joint member, a linking assembly disposed between and pivoted to the upper and lower joint members, and a joint friction member that includes a first friction piece adjustably secured to the lower joint member and having a top curved face, and a second friction piece connected to the linking assembly and having a bottom curved face which conforms to and which is slidable on the top curved face of the first friction piece so as to provide a frictional force against movement of the upper joint member relative to the lower joint member.

### CROSS REFERENCE TO RELATED APPLICATION

This application claims priority of Taiwan patent Application No. 090209399, filed on Jun 6, 2001.

### BACKGROUND OF THE INVENTION

1. Field of the invention

This invention relates to an artificial knee joint, more particularly to an artificial knee joint with a joint friction member for ensuring smooth operation of the knee joint.

2. Description of the related art

FIG. 1 illustrates a conventional artificial knee joint for an above-knee amputee. The artificial knee joint includes an upper joint member 81 that is adapted to be connected to a thigh support (not shown) a lower joint member 83 that is adapted to be connected to a leg support (not shown), and a linking assembly 85 that interconnects and that is pivoted to the upper and lower joint members 81, 83 and that includes upper and lower links 812, 811 which are vertically aligned along a vertical line (not visible) when an associated one of the user's feet (not shown) touches the ground while walking and which are not aligned along the vertical line when the associated one of the user's feet (not shown) is lifted from the ground, which is a consequence of flexing of the upper joint member 81 relative to the lower joint member 83. A cushioning pad 813 is disposed underneath the lower link 811 and is deformable when pressed by the lower link 811 so as to absorb shock when the associated one of the user's feet touches the ground upon walking.

The conventional artificial knee joint is disadvantageous in that it does not include a speed controlling device to control the speed of rotation of the lower link 811. As a consequence, there is a tendency for the lower link 811 to be not able to rotate smoothly upon walking. Moreover, the cushioning effect provided by the cushioning pad 813 is significantly reduced when the user walks on a slope due to flexing of the upper link 812 relative to the lower link 811, i.e. the upper and lower links 812, 811 are not aligned along the vertical line.

### SUMMARY OF THE INVENTION

Therefore, the object of the present invention is to provide an artificial knee joint that is capable of overcoming the aforementioned drawbacks.

Accordingly, an artificial knee joint of this invention comprises: an upper joint member; a lower joint member disposed below the upper joint member; a linking assembly disposed between and pivoted to the upper and lower joint members so as to permit the upper joint member to be movable relative to the lower joint member between a first position, in which the upper joint member is vertically aligned with the lower joint member along a vertical line, and a second position, in which the upper joint member is not vertically aligned with the lower joint member along the vertical line; and a joint friction member including a first friction piece that is adjustably secured to the lower joint member and that has a top curved face, and a second friction piece that is connected to the linking assembly and that has a bottom curved face which conforms to and which is slidable on the top curved face of the first friction piece so as to provide a frictional force against movement of the upper joint member between the first and second positions.

### BRIEF DESCRIPTION OF THE DRAWINGS

In drawings which illustrate an embodiment of the invention,

FIG. 1 illustrates a conventional artificial knee joint;

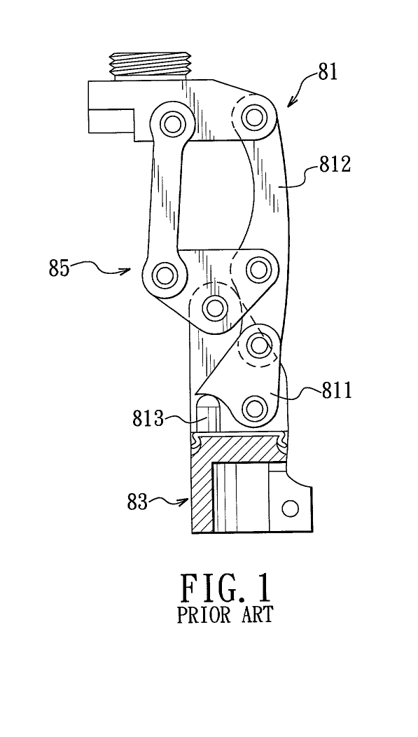

FIG. 2 is an exploded view of an artificial knee joint embodying this invention;

FIG. 3 is a side view of the artificial knee joint of FIG. 2;

FIG. 4 is a partly cross-sectional view to illustrate operation of a joint friction member of the artificial knee joint of FIG. 2;

FIG. 5 is another partly cross-sectional view to illustrate operation of a cushioning unit of the artificial knee joint of FIG. 2; and

FIG. 6 is yet another partly cross-sectional view to illustrate operation of a returning unit of the artificial knee joint of FIG. 2.

### DETAILED DESCRIPTION OF THE PREFERRED EMBODIMENT

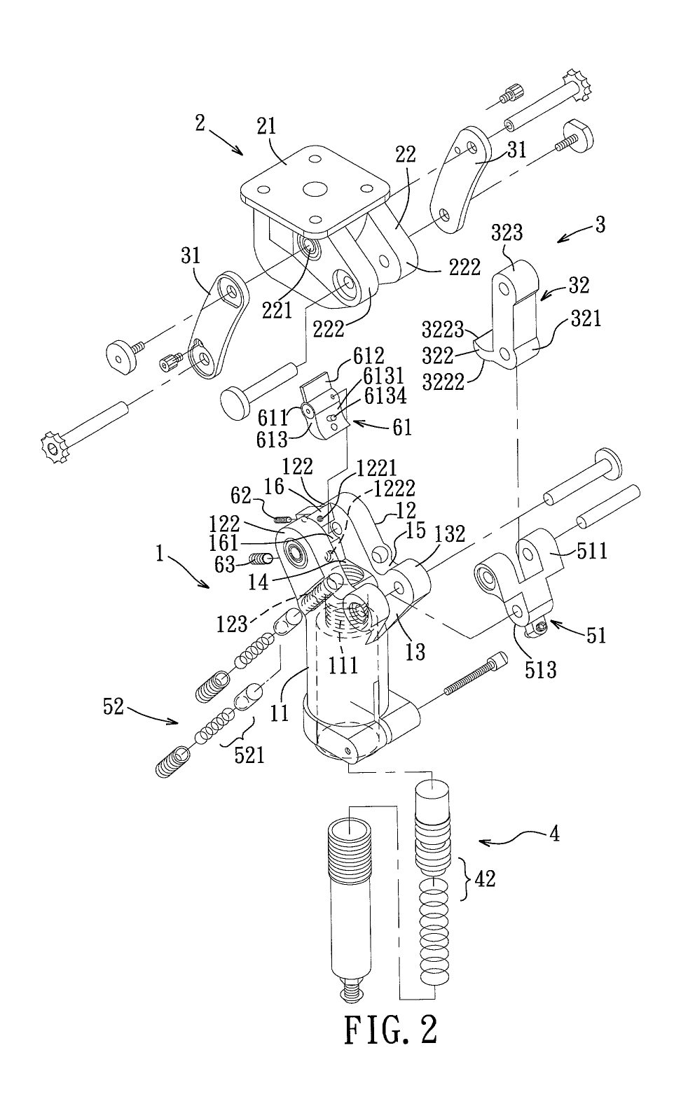

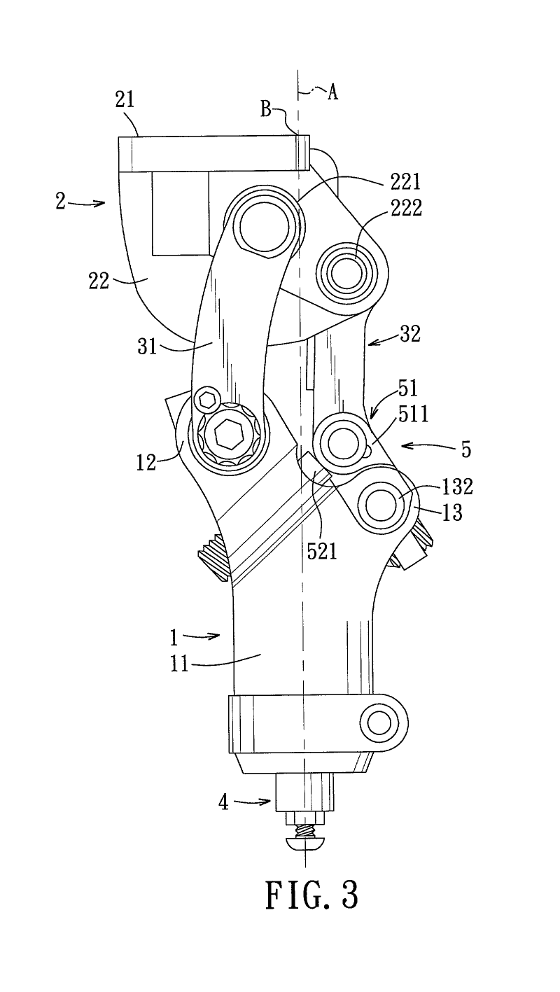

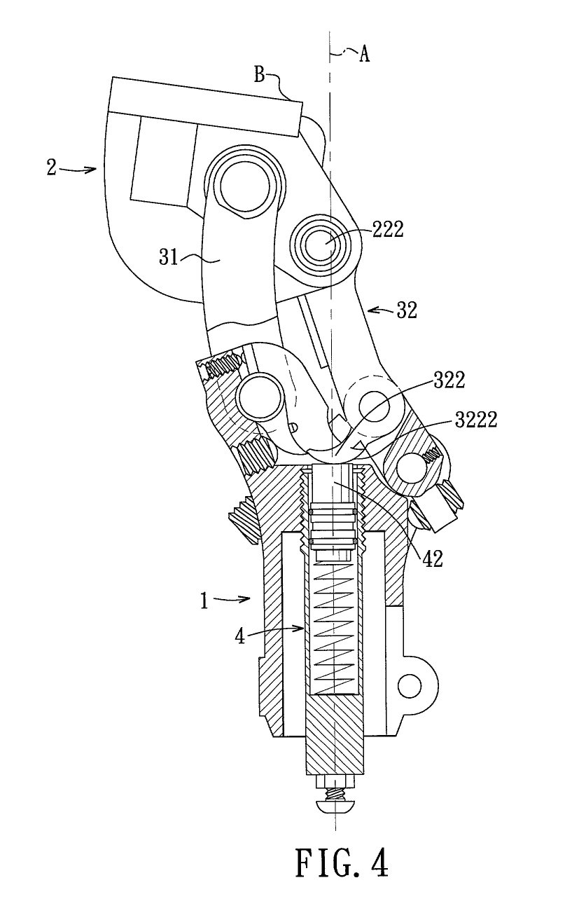

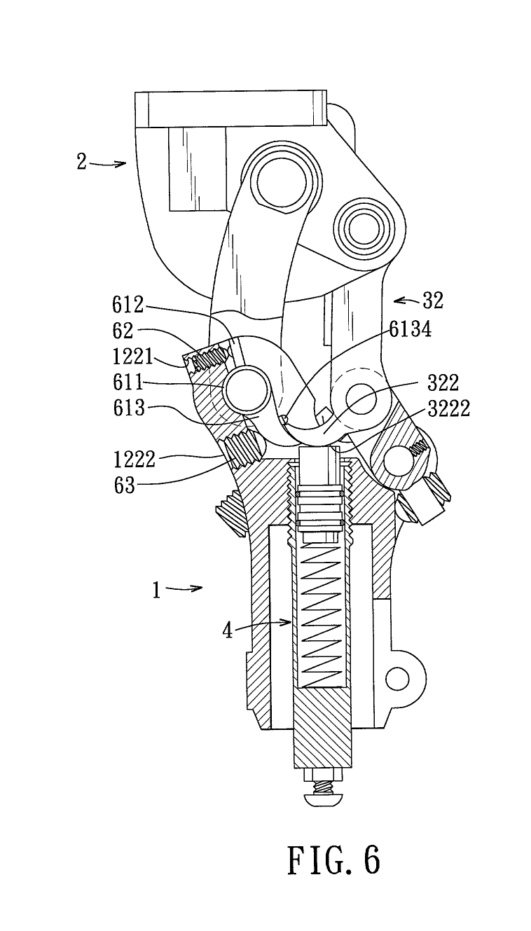

FIGS. 2 to 6 illustrate a preferred embodiment of an artificial knee joint of this invention for an above-knee amputee. The artificial knee joint includes: an upper joint member 2 that is adapted to be connected to a thigh support (not shown) ; a lower joint member 1 that is disposed below the upper joint member 2 and that is adapted to be connected to a leg support (not shown); a linking assembly 3 disposed between and pivoted to the upper and lower joint members 2, 1 so as to permit the upper joint member 2 to be movable relative to the lower joint member 1 between a first position, in which the upper joint member 2 is vertically aligned with the lower joint member 1 along a vertical line (A) (see FIG. 3), which passes through a center line of the lower joint member 1 and a point (B) on the upper joint member 2, and a second position, in which the upper joint member 2 is not vertically aligned with the lower joint member 1 along the vertical line (A) (see FIG. 4); and a joint friction member including a first friction piece 61 that is adjustably secured to the lower joint member 1 and that has a top curved face 6131, and a second friction piece 322 that is connected to the linking assembly 3 and that has a bottom curved face 3222 which conforms to and which is slidable on the top curved face 6131 of the first friction piece 61 (see FIGS. 4 and 6) so as to provide a frictional force against movement of the upper joint member 2 between the first and second positions, so as to minimize uncontrolled movement of the upper joint member 2, and so as to ensure smooth operation of the artificial knee joint upon walking.

The second friction piece 322 further has a front edge 3223 that extends toward the first friction piece 61. The first friction piece 61 includes a stopper 6134 that protrudes from the top curved face 6131 and that is anterior to the front edge 3223 of the second friction piece 322 for engaging the latter (see FIG. 6) so as to limit further sliding movement of the second friction piece 322 on the top curved face 6131 of the first friction piece 61 when the upper joint member 2 moves from the second position to the first position.

The lower joint member 1 includes a forked head that has a pair of spaced apart front arms 12 with front pivot ends 122, a pair of spaced apart rear arms 13 with rear pivot ends 132 which are positioned at a level lower than that of the front pivot ends 122, and a central recess 14 confined by the front and rear arms 12, 13. Each of the front arms 12 and a respective one of the rear arms 13 define a side recess 15 therebetween which extends from a respective one of the front pivot ends 122 to a respective one of the rear pivot ends 132.

The linking assembly 3 includes rear upper and lower links 32, 51. The rear lower link 51 has a lower pivot end 513 that is disposed between and that is pivoted to the rear pivot ends 132 of the rear arms 13, and a pair of spaced apart upper pivot ends 511 that project from two opposite sides of the lower pivot end 513 and that are disposed immediately above the side recesses 15 in the forked head. The rear upper link 32 has a bottom pivot end 321 which is disposed between and which is pivoted to the upper pivot ends 511 of the rear lower link 51.

The first and second friction pieces 61, 322 are disposed in the central recess 14. The second friction piece 322 extends curvedly and frontwardly from the bottom pivot end 321 of the rear upper link 32 to the front edge 3223 to define the bottom curved face 3222. The first friction piece 61 includes a middle pivot portion 611 that is disposed between and that is pivoted to the front pivot ends 122 of the front arms 12, and a rear portion 613 that extends curvedly and downwardly from the middle pivot portion 611 toward the second friction piece 322 to define the top curved face 6131.

The forked head further has a cross-plate 16 that interconnects the front arms 12 and that is formed with a groove 161 for receiving a portion of the middle pivot portion 611 of the first friction piece 61, and upper and lower threaded holes 1221, 1222 disposed at two opposite sides of the groove 161. The first friction piece 61 further has a front portion 612 that extends from the middle pivot portion 611 in a direction opposite to that of the rear portion 613. A friction adjusting unit includes upper and lower screw rods 62, 63 that extend through and that engage threadedly the upper and lower threaded holes 1221, 1222, respectively, and that have rear ends abutting against the rear portion 613 of the first friction piece 61 (see FIGS. 4 and 6) so as to provide an optimum frictional force when the bottom curved face 3222 of the second friction piece 322 slides on the top curved face 6131 of the first friction piece 61.

The lower joint member 1 further includes a hollow shank 11 that extends downwardly from the forked head and that defines an inner space 111 which is in spatial communication with the central recess 14. A returning unit 4 includes a first spring-loaded member 42 disposed movably in the inner space 111 in the shank 11 and extending into the central recess 14 to abut against the second friction piece 322 (see FIGS. 4 and 6) so as to provide an urging force to the rear upper link 32 for restoring of the upper joint member 2 from the second position to the first position.

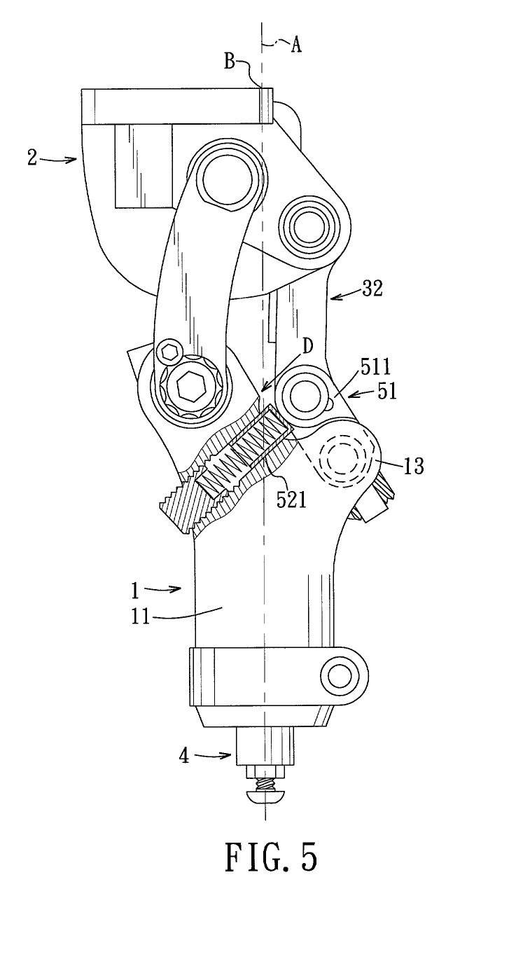

Each of the front arms 12 has opposite front and rear sides, and is formed with a retaining hole 123 that extends from the front side to the rear side to communicate spatially with a respective one of the side recesses 15. A cushioning unit 52 includes a pair of second spring-loaded members 521, each of which is disposed movably in a respective one of the retaining holes 123 in the front arms 12 and each of which extends into a respective one of the side recesses 15 to abut against a respective one of the upper pivot ends 511 of the rear lower link 51 (see FIG. 5) so as to provide a cushioning effect when the rear lower link 51 turns frontwardly and downwardly to press against the second spring-loaded members 521 in directions (indicated as reference mark “D” in FIG. 5) that are inclined relative to the vertical line (A) and so as to provide an urging force to the rear upper link 32 via the rear lower link 51 for facilitating frontward movement of the upper joint member 2 from the first position to the second position.

The upper joint member 2 includes a top plate 21 and a pair of spaced apart legs 22 that project downwardly from the top plate 21 and that have front and rear pivot ends 221, 222. The rear upper link 32 further has a top pivot end 323 that is disposed between and that is pivoted to the rear pivot ends 222 of the legs 22. The linking assembly 3 further includes a pair of side links 31 that are pivoted to the front pivot ends 221 of the legs 22 and the front pivot ends 122 of the front arms 12.

Due to the presence of the joint friction member, i.e. the first and second friction pieces 61, 322, and the inclined design of the cushioning unit 52 relative to the vertical line (A), the drawbacks associated with the prior art can be eliminated.

With the invention thus explained, it is apparent that various modifications and variations can be made without departing from the spirit of the present invention. It is therefore intended that the invention be limited only as recited in the appended claims.

I claim:

1. An artificial knee joint comprising:
an upper joint member;
a lower joint member disposed below said upper joint member;
a linking assembly disposed between and pivoted to said upper and lower joint members so as to permit said upper joint member to be movable relative to said lower joint member between a first position, in which said upper joint member is vertically aligned with said lower joint member along a vertical line, and a second position, in which said upper joint member is not vertically aligned with said lower joint member along said vertical line; and
a joint friction member including a first friction piece that is adjustably secured to said lower joint member and that has a top curved face, and a second friction piece that is connected to said linking assembly and that has a bottom curved face which conforms to and which is slidable on said top curved face of said first friction piece so as to provide a frictional force against movement of said upper joint member between said first and second positions.

2. The artificial knee joint of claim 1, wherein said second friction piece further has a front edge that extends toward said first friction piece, said first friction piece including a stopper that protrudes from said top curved face and that is anterior to said front edge of said second friction piece for engaging said front edge of said second friction piece so as to limit further sliding movement of said second friction piece on said top curved face of said first friction piece when said upper joint member moves from said second position to said first position.

3. The artificial knee joint of claim 2, wherein said lower joint member includes a forked head that has a pair of spaced apart front arms with front pivot ends, a pair of spaced apart rear arms with rear pivot ends which are positioned at a level lower than that of said front pivot ends, and a central recess confined by said front and rear arms, each of said front arms and a respective one of said rear arms defining a side recess therebetween which extends from a respective one of said front pivot ends to a respective one of said rear pivot ends, said linking assembly including rear upper and lower links, said rear lower link having a lower pivot end that is disposed between and that is pivoted to said rear pivot ends of said rear arms, and a pair of spaced apart upper pivot ends that project from two opposite sides of said lower pivot end and that are disposed immediately above said side recesses in said forked head, said rear upper link having a bottom pivot end which is disposed between and which is pivoted to said upper pivot ends of said rear lower link, said first and second friction pieces being disposed in said central recess, said second friction piece extending curvedly and frontwardly from said bottom pivot end of said rear upper link to said front edge to define said bottom curved face, said first friction piece including a middle pivot portion that is disposed between and that is pivoted to said front pivot ends of said front arms, and a rear portion that extends curvedly and downwardly from said middle pivot portion toward said second friction piece to define said top curved face.

4. The artificial knee joint of claim 3, wherein said forked head further has a cross-plate that interconnects said front arms and that is formed with a groove for receiving a portion of said middle pivot portion of said first friction piece, and upper and lower threaded holes disposed at two opposite sides of said groove, said first friction piece further having a front portion that extends from said middle pivot portion in a direction opposite to that of said rear portion, said artificial knee joint further comprising a friction adjusting unit which includes upper and lower screw rods that extend through and that engage threadedly said upper and lower threaded holes, respectively, and that have rear ends abutting against said rear portion of said first friction piece when said bottom curved face of said second friction piece slides on said top curved face of said first friction piece.

5. The artificial knee joint of claim 4, wherein said lower joint member further includes a hollow shank that extends downwardly from said forked head and that defines an inner space which is in spatial communication with said central recess, said artificial knee joint further comprising a returning unit that includes a first spring-loaded member disposed movably in said inner space in said shank and extending into said central recess to abut against said second friction piece so as to provide an urging force to said rear upper link for restoring said upper joint member from said second position to said first position.

6. The artificial knee joint of claim 5, wherein each of said front arms has opposite front and rear sides, and is formed with a retaining hole that extends from said front side to said rear side to communicate spatially with a respective one of said side recesses, said artificial knee joint further comprising a cushioning unit that includes a pair of second spring-loaded members, each of which is disposed movably in a respective one of said retaining holes in said front arms and each of which extends into a respective one of said side recesses to abut against a respective one of said upper pivot ends of said rear lower link so as to provide a cushioning effect when said rear lower link turns frontwardly and downwardly to press against said second spring-loaded members in directions that are inclined relative to said vertical line and so as to provide an urging force to said rear upper link via said rear lower link for facilitating frontward movement of said upper joint member from said first position to said second position.

7. The artificial knee joint of claim 6, wherein said upper joint member includes a top plate and a pair of spaced apart legs that project downwardly from said top plate and that have front and rear pivot ends, said rear upper link further having a top pivot end that is disposed between and that is pivoted to said rear pivot ends of said legs, said linking assembly further including a pair of side links that are pivoted to said front pivot ends of said legs and said front pivot ends of said front arms.
